(19) United States
(12) Patent Application Publication
(10) Pub. No.: US 2023/0282359 A1
(43) Pub. Date: Sep. 7, 2023

(54) DATA PROCESSING SYSTEM AND DATA PROCESSING METHOD

(71) Applicant: Hitachi, Ltd., Tokyo (JP)

(72) Inventors: Takahiro NAKAMURA, Tokyo (JP); Takashi TAKEMOTO, Tokyo (JP); Miaomei LEI, Tokyo (JP); Keisuke TERADA, Tokyo (JP)

(21) Appl. No.: 18/100,137

(22) Filed: Jan. 23, 2023

(30) Foreign Application Priority Data
Feb. 1, 2022 (JP) 2022-014477

(51) Int. Cl. G16H 50/30

(52) U.S. Cl. CPC G16H 50/30

(57) ABSTRACT

Health data is made available to a private business operator to improve health of a resident. A data processing system includes a storage device accessible by an arithmetic processing device; a reception unit configured to receive input data; an information conversion unit configured to convert the input data; a health prediction model generated based on nutrient information and an outcome; and an output unit configured to output information of a subject based on a prediction result generated by the health prediction model. The reception unit receives the input data relating to a customer of a business operator, the information conversion unit converts the input data into customer nutrient information indicating a nutrient ingested by the customer, the health prediction model predicts health based on the customer nutrient information, and the output unit outputs advice for the health of the subject to the business operator based on the prediction result.

### CLAIM OF PRIORITY

The present application claims priority from Japanese patent application No. 2022-14477 filed on Feb. 1, 2022, the content of which is hereby incorporated by reference into this application.

### BACKGROUND OF THE INVENTION

### 1. Field of the Invention

The present invention relates to a data processing system, and more particularly, to a system that provides meaningful information based on health data to a private business operator.

### 2. Description of the Related Art

For the purpose of improving the health of a resident, local governments and the like are conducting health projects that collect and analyze health data from the resident. Such health projects are sometimes conducted as cohort studies in collaboration with universities, research institutes, and companies. The health data from the resident collected in the cohort studies are analyzed and utilized to identify factors such as disease and health indicators, which is the purpose of the cohort studies.

The following related art is described as a background art in the present technical field. JP-A-2020-135720 (Patent Literature 1) discloses a shopping assistance system including a management terminal that manages at least information on food sales, a user terminal owned by a user, and a server device that can communicate with the management terminal and the user terminal. The management terminal generates digitized purchase information based on information on food including the price of each food purchased by the user, and transmits the digitized purchase information to the server device. The server device includes a food information acquisition unit that acquires food information including information on the price and quantity exchange ratio for each food, estimates an amount of each food ingested by the user and family members of the user based on personal information of the user and the family members, the price of each food indicated by the digitized purchase information received from the management terminal, and the acquired food information in at least a part of the foods purchased by the user, allocates the estimated amount of each food for each person of the user and the family members and for each day from the purchase date, analyzes food nutrition information regarding nutritive values for each type of nutrients possessed by the food estimated to be ingested by the user and the family members for each day in units of individual based on the result of the allocation, generates nutrition deviation information of the user and the family members indicating a deviation of nutrients for each day, and transmits the generated nutrition deviation information to the user terminal. The user terminal receives the nutrition deviation information transmitted from the server device and outputs the received nutrition deviation information.

It is difficult for a private enterprise to utilize the health data held by the local governments from the viewpoint of personal information protection. If a private business operator can utilize the health data, it can be expected to improve the health of the resident by providing various health services. For this purpose, from the viewpoint of personal information protection and ethics, it is necessary to obtain consent of the subject resident for each purpose. Since it is not easy to obtain the consent from tens of thousands of people for each purpose, there is a need to present meaningful information to the private business operator without direct access to the health data, and to enable the private business operator to utilize the knowledge obtained from the cohort studies.

### SUMMARY OF THE INVENTION

A representative example of the invention disclosed in the present application is as follows. That is, a data processing system includes: an arithmetic device configured to execute arithmetic processing; a storage device accessible by the arithmetic device; a reception unit configured to receive input data; an information conversion unit configured to convert the input data; a health prediction model generated based on nutrient information and an outcome; and an output unit configured to output information of a subject based on a prediction result generated by the health prediction model. The reception unit receives the input data relating to a customer of a business operator, the information conversion unit converts the input data into customer nutrient information indicating a nutrient ingested by the customer, the health prediction model predicts health based on the customer nutrient information, and the output unit outputs advice for the health of the subject to the business operator based on the prediction result.

According to an aspect of the invention, the health of a resident can be improved. Problems, configurations, and effects other than those described above will be clarified with the following description of embodiments.

### BRIEF DESCRIPTION OF THE DRAWINGS

FIG. 1 is a block diagram showing a physical configuration of a data processing system according to a first embodiment.

FIG. 2 is a configuration diagram of the data processing system according to the first embodiment.

FIG. 3 is a diagram in which nutrient ingestion information of each test subject in an analysis result database according to the first embodiment is mapped in a multidimensional space.

FIG. 4 is a diagram showing a configuration example of the analysis result database according to the first embodiment.

FIG. 5 is a configuration diagram of a data processing system according to a second embodiment.

FIG. 6 is a configuration diagram of a purchase information correction unit according to the second embodiment.

FIG. 7 is a diagram showing an example of thresholds in a factor determination unit according to the second embodiment.

FIG. 8 is a configuration diagram of a data processing system according to a third embodiment.

FIG. 9 is a configuration diagram of a nutrient information comparison and correction unit 323 according to the third embodiment.

### DESCRIPTION OF THE PREFERRED EMBODIMENTS

In the following embodiments, the description may be divided into a plurality of sections or embodiments if necessary for convenience. Unless otherwise specified, the sections or embodiments are not independent of each other, but have a relation in which one section or embodiment is a modification, detailed description, supplementary description, or the like of a part or all of another section or embodiment. In the following embodiments, when a reference is made to the number of elements, and the like (including a count, a numeric value, an amount, a range, and the like), unless otherwise specified and limited theoretically apparently to a specific number and the like, the number is not limited to the specific number and may be either equal to or larger than or equal to or less than the specific number.

Further, in the following embodiments, it is needless to say that elements (including element steps and the like) are not always indispensable unless otherwise stated or except a case where the components are apparently indispensable in principle. Similarly, in the following embodiments, shapes, positional relation, or the like of the elements or the like include those substantially approximate or similar to the shapes or the like unless otherwise particularly specified or considered to be obviously excluded in principle. The same applies to the numerical value and the range.

Hereinafter, embodiments of the invention will be described in detail with reference to the drawings. The same elements are denoted by the same reference signs in principle throughout all the drawings for showing the embodiments, and the repetitive description thereof is omitted.

### First Embodiment

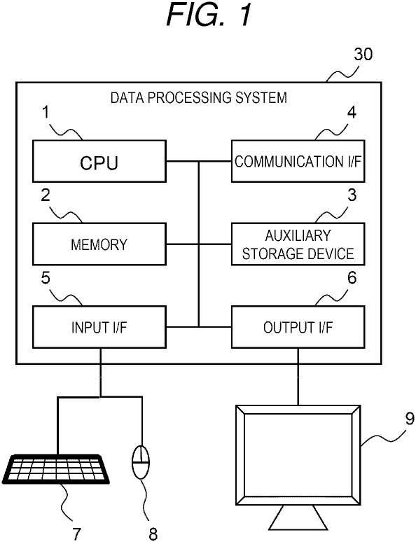

FIG. 1 is a block diagram showing a physical configuration of a data processing system 30 according to a first embodiment of the invention.

The data processing system 30 according to the present embodiment is implemented by a computer including a processor (CPU) 1, a memory 2, an auxiliary storage device 3, and a communication interface 4. The data processing system 30 may include an input interface 5 and an output interface 6.

The processor 1 is an arithmetic device that executes a program stored in the memory 2. The processor 1 executes various programs to implement functional units (a correction unit 320, an intervention proposal and effect determination unit 330, and an input and output unit 340) provided by the data processing system 30. A part of the processing performed by the processor 1 executing the programs may be executed by another arithmetic device (for example, hardware such as an ASIC or an FPGA) .

The memory 2 includes a ROM, which is a non-volatile storage element, and a RAM, which is a volatile storage element. The ROM stores an invariable program (for example, a BIOS) or the like. The RAM is a high-speed and volatile storage element such as a dynamic random access memory (DRAM), and temporarily stores the programs to be executed by the processor 1 and data to be used when the programs are executed.

The auxiliary storage device 3 is, for example, a large-capacity, non-volatile storage device such as a magnetic storage device (HDD) or a flash memory (SSD), and constitutes a database unit 310. In addition, the auxiliary storage device 3 stores data (for example, an analysis result database 311) to be used when the processor 1 executes the programs, and the programs to be executed by the processor 1. That is, the programs are read from the auxiliary storage device 3, loaded into the memory 2, and executed by the processor 1, thereby implementing functions of the data processing system 30.

The communication interface 4 is a network interface device that controls communication with other devices (for example, a terminal 500) in accordance with a predetermined protocol.

The input interface 5 is an interface to which an input device such as a keyboard 7 or a mouse 8 is connected and which receives an input from an operator. The output interface 6 is an interface to which an output device such as a display device 9 or a printer (not shown) is connected and which outputs execution results of the programs in a format that can be visually recognized by the operator. A user terminal (not shown) connected to the data processing system 30 via a network may provide the input device and the output device. In this case, the data processing system 30 may have a function of a web server, and the user terminal may access the data processing system 30 using a predetermined protocol (for example, http).

The programs to be executed by the processor 1 are provided to the data processing system 30 via a removable medium (CD-ROM, flash memory, and the like) or a network, and are stored in the non-volatile auxiliary storage device 3 which is a non-transitory storage medium. Therefore, the data processing system 30 may include an interface that reads data from the removable medium.

The data processing system 30 may be a computer system implemented on one physical computer or on a plurality of logical or physical computers, and may be operated on a virtual computer constructed on a plurality of physical computer resources. For example, the correction unit 320, the intervention proposal and effect determination unit 330, and the input and output unit 340 may operate on separate physical or logical computers, or may operate on one physical or logical computer by combining a plurality of computers.

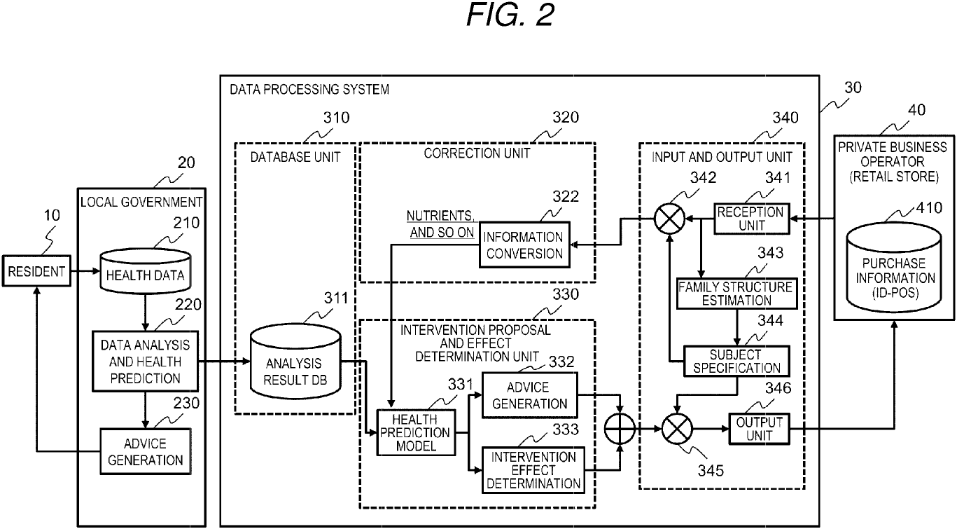

FIG. 2 is a configuration diagram of the data processing system 30 according to the first embodiment of the invention.

The data processing system 30 according to the first embodiment includes the database unit 310, the correction unit 320, the intervention proposal and effect determination unit 330, and the input and output unit 340. The input and output unit 340 includes: a reception unit 341 that receives purchase information (that is, ID-POS data) from a private business operator 40 such as a retail store; a family structure estimation unit 343 that estimates a family structure for each piece of received purchase data, a subject specification unit 344 that specifies a subject from the estimated family structure and outputs information for specifying the subject such as the family structure, age, and gender; a purchase information selection unit 342 that selects purchase information of the specified subject; a result selection unit 345 that selects information of the specified subject from an output result of the intervention proposal and effect determination unit 330; and an output unit 346 that outputs information of the subject and advice on product sales.

The private business operator 40 advertises to encourage the purchase of a product suitable for the subject in accordance with the advice output from the output unit 346 by using a POS system.

The database unit 310 includes the analysis result database 311. The analysis result database 311 stores an analysis result of health data 210 held by a local government or the like provided by a local government health data system 20. The correction unit 320 includes an information conversion unit 322 that converts the purchase information into nutrient information. The intervention proposal and effect determination unit 330 includes a health prediction model 331, an advice generation unit 332, and an intervention effect determination unit 333.

The local government health data system 20 collects the health data 210 of a resident 10, and includes a data analysis and health prediction unit 220 that analyzes the health data 210 and predicts a future health condition, and an advice generation unit 230 that generates advice to the resident 10 based on a prediction result of the future health condition.

When the input and output unit 340 receives the selected purchase information, the correction unit 320 converts the received purchase information into the nutrient information. The health prediction model 331 predicts the health of the subject using the nutrient information. The advice generation unit 332 generates advice based on health prediction and subject information. The intervention effect determination unit 333 determines an effect caused by a time-series change of the purchase information based on the time-series change of the purchase information and the like. The intervention effect determination unit 333 may be implemented by, for example, a model base. The input and output unit 340 selects the subject of the generated advice and an intervention effect, and outputs the generated advice and the intervention effect to the selected subject.

The health prediction model 331 may be generated from the health data 210 based on machine learning or the like, but it is difficult to utilize the health data 210 held by the local government as described above. In the present embodiment, a result generated by statistically analyzing the health data 210 and anonymized data are stored in the analysis result database 311, and the health prediction model 331 is created based on the analysis result database 311. Since the analysis result database 311 stores statistical information in which personal information is not included and the anonymized data, it is not necessary to obtain consent from the resident or the like.

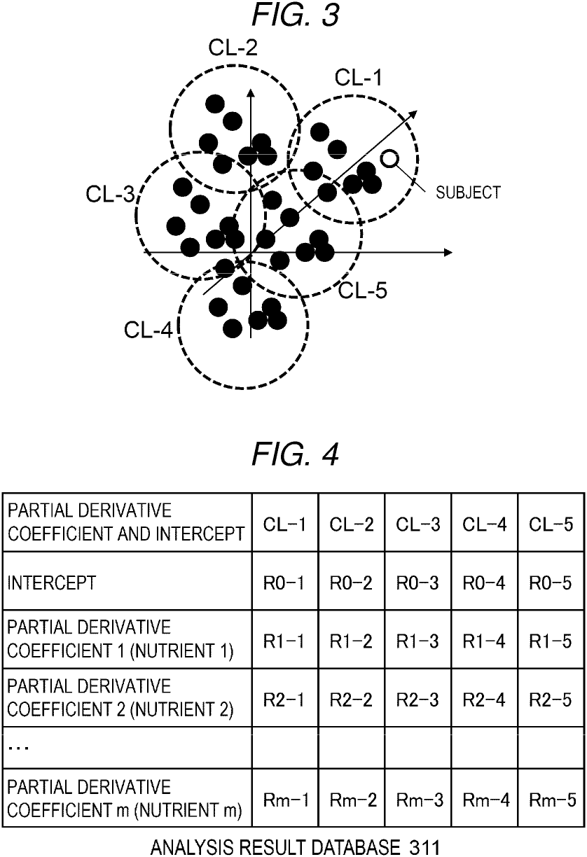

Next, the analysis result database 311 will be described. First, as shown in FIG. 3, nutrient ingestion information of each test subject in the health data 210 is mapped to an N-dimensional space (N is a positive integer) and clustered. For the nutrient ingestion information, the number of dimensions may be reduced by principal component analysis (PCA) or the like, nutrients having strong relevance may be collected, and a calculation cost may be reduced. Next, for each cluster (CL-1, CL-2, CL-3, CL-4, CL-5 in FIG. 3), a partial regression coefficient and an intercept of each nutrient of a multiple regression model for outcomes are calculated. The outcomes are factors that influence the health of the subject. For example, the outcomes for pregnant women include childbirth weight and diversity of gut microbiota, while the outcomes for the elderly include frailty risk, dementia risk, and heart disease risk. Instead of the multiple regression model, a learned neural network may be used.

The health prediction model 331 determines which cluster the nutrient information of the subject belongs to, based on the nutrient information of the subject, and applies the multiple regression model of the cluster to which the nutrient information belongs.

FIG. 4 is a diagram showing an example of a partial regression coefficient and an intercept in each cluster with respect to the outcome recorded in the analysis result database 311, and shows the partial regression coefficient and the intercept of each nutrient to be ingested in the multiple regression model when an intestinal flora diversity is set to the outcome. In the analysis result database 311, cluster information (surface coordinate information, center of gravity information, and the like of each cluster) in FIG. 3 and information on the partial regression coefficient and the intercept for each outcome in FIG. 4 are registered. The partial regression coefficient and the intercept are explanatory variables of the multiple regression model, and the outcomes are objective variables of the multiple regression model.

The multiple regression model can be created by a procedure described later. First, the nutrient ingestion information of the subject is mapped, and a corresponding cluster is specified. When mapped coordinates are located in a gap between the clusters or outside the clusters and do not belong to any cluster, it is possible to avoid a situation in which the mapped coordinates do not belong to any cluster by determining that the mapped coordinates belong to a cluster whose center of gravity is the closest to the mapped coordinates. The cluster may be specified based on the mapped coordinates and center of gravity information of each cluster such that the mapped coordinates are determined to belong to a cluster having the closest distance to the center of gravity. In this case, since the surface coordinate information of the cluster is unnecessary, the data capacity and the calculation cost can be reduced. The multiple regression model for predicting an outcome is created based on the partial regression coefficient and the intercept of the specified cluster. When the multiple regression model is created for all elements, for example, the model is incorrect for elements that are quadratic functions, but by dividing into clusters, the possibility that linear approximation can be performed in the clusters is increased, so that a more accurate model can be created, and appropriate advice can be generated in consideration of diversity.

The data processing system 30 according to the first embodiment includes: an arithmetic device (the processor 1) that executes arithmetic processing; a storage device (the memory 2, the auxiliary storage device 3) accessible by the processor 1; the reception unit 341 that receives input data; an information conversion unit 322 that converts the input data; the health prediction model 331 generated based on nutrient information and an outcome; and the output unit 346 that outputs information of a subject based on a prediction result generated by the health prediction model 331. The reception unit 341 receives the input data relating to a customer of a business operator, the information conversion unit 322 converts the input data into customer nutrient information indicating nutrients ingested by the customer of the business operator, the health prediction model 331 predicts health based on the customer nutrient information, and the output unit 346 outputs advice for the health of the subject or information of the subject to the business operator based on the prediction result. Therefore, the health of the resident can be improved by the action of the private business operator, and medical cost can be reduced. The private business operator can utilize knowledge obtained from cohort studies without directly accessing the health data held by the local government. This data can be used to develop health services, it is expected to increase the number of users by expanding services and optimize inventory by purchasing forecasts, and the profit can be improved.

### Second Embodiment

In the first embodiment described above, the nutrient information obtained from the purchase information is treated as the total ingestion nutrient of the subject during a certain period of time. However, in reality, since the subject purchases food from a plurality of retail stores and eats out, the subject also ingests nutrients that are not included in the purchase information. Therefore, there is a problem that accurate ingestion nutrients cannot be obtained only by purchase information of a specific retail store. In a second embodiment, a purchase information correction unit 321 is provided in the correction unit 320 to solve this problem. In the second embodiment, differences from the first embodiment described above will be mainly described, and descriptions of the same configurations and functions as those of the first embodiment will be omitted.

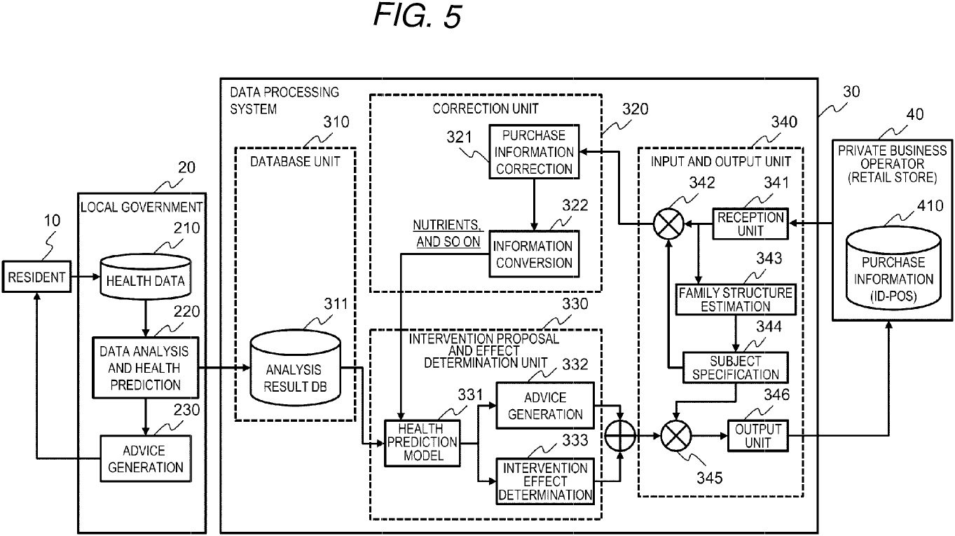

FIG. 5 is a configuration diagram of the data processing system 30 according to the second embodiment.

The data processing system 30 according to the second embodiment includes the database unit 310, the correction unit 320, the intervention proposal and effect determination unit 330, and the input and output unit 340. The correction unit 320 includes the purchase information correction unit 321 and the information conversion unit 322 that converts the purchase information into nutrient information.

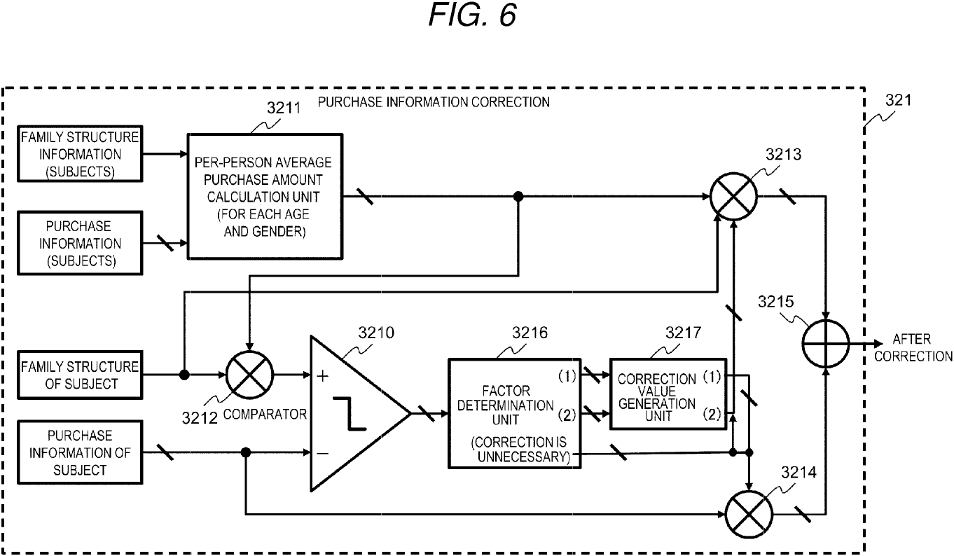

FIG. 6 is a configuration diagram of the purchase information correction unit 321 according to the second embodiment.

The purchase information correction unit 321 includes a per-person average purchase calculation unit 3211, a comparator 3210, a factor determination unit 3216, a correction value generation unit 3217, multipliers 3212, 3213, and 3214, and an adder 3215.

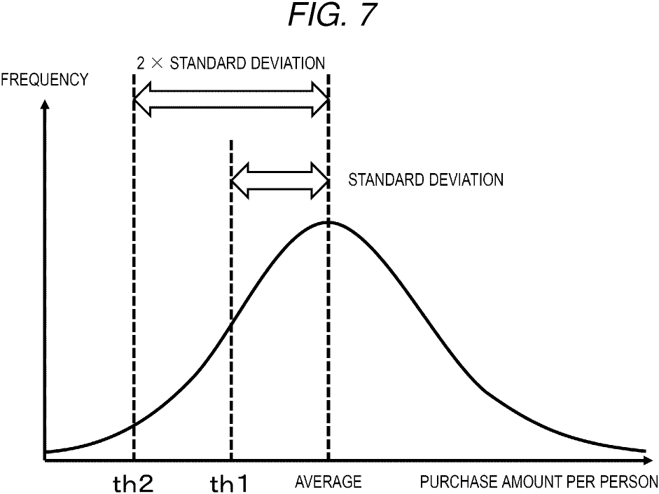

The per-person average purchase calculation unit 3211 calculates a reference value using purchase information of a plurality of subjects and family structure information. The family structure information may be provided from the subject or may be estimated from the purchase information. The per-person average purchase calculation unit 3211 classifies the input purchase information for each purchased item by a classifier, and calculates, as the reference value, an average purchase amount per person for each purchased item based on the purchase information for each purchased item and the family structure information. The purchased items are, for example, items such as cereals, meat, fish, soybean products, dairy products, vegetables, taros, seaweed, fruits, fats and oils, and seasonings, and are generally distributed as shown in FIG. 7. The average purchase amount is the purchase price or purchase weight.

There are several possible factors for the small purchase amount of the subject. For example, (1) the subject also purchases from other retail stores; (2) the subject purchases some foods from other retail stores (vegetables purchased from greengrocers, rice purchased online, etc.); (3) the subject does not purchase specific food items for hobbies or tastes. The factor (3) does not need to be corrected, but in order to correct the decrease in the purchase amount due to the factor (1) and the factor (2), the comparator 3210 first compares the calculated reference value with the purchase information of the subject. Based on the calculated reference value and the family structure of the subject, purchase information serving as a reference is calculated and compared with the purchase information of the subject. In the case of the factor (1), the purchase amount is reduced as a whole. Therefore, the factor determination unit 3216 determines that the factor is the factor (1) when a ratio (k1) of an average value of the purchase amount of each purchased item in the purchase information of the subject to the purchase information serving as a reference is equal to or less than a threshold value 1 (th1). The threshold value 1 (th1) is a statistically significant threshold value by employing a value obtained by subtracting a standard deviation from the average value of the purchase information of the plurality of subjects used in the calculation of the reference value. The purchase information of the subject determined to be the factor (1) is corrected by multiplying all the items by th1/k1. Specifically, the correction value generation unit 3217 generates a correction value, and the multiplier 3214 multiplies the generated correction value by the purchase information of the subject and outputs the result. By such correction, it is possible to correct the decrease in the purchase amount due to the factor (1) and prevent the purchase amount from being discontinuous with respect to k1. Since when k1 is extremely small, an error due to the correction may increase, when k1 is equal to or less than a certain threshold value 2 (th2), the correction cannot be performed, and when k1 is excluded from the subsequent process, it is possible to prevent the health prediction with low accuracy. The threshold value 2 (th2) is a statistically significant threshold value by employing a value obtained by subtracting 2 × standard deviation from the average value of the purchase information of the plurality of subjects used in the calculation of the reference value.

Although it is difficult to distinguish between the factor (2) and the factor (3), the factor (2) can be determined when the grains and the vegetables are extremely small. When the purchase amount of cereals or vegetables is equal to or less than a predetermined threshold 3 (th3) regardless of the factor (1), the factor determination unit 3216 determines that the factor is factor (2). The threshold value 3 (th3) is a statistically significant threshold value by employing a value obtained by subtracting 2 × standard deviation from the average value of the purchase information of the plurality of subjects used in the calculation of the reference value. By correcting the purchase amount of the grains and the vegetables having the threshold value 3 or less to average purchase information × th3/average value, the continuity is ensured while preventing excessive correction. Specifically, the correction value generation unit 3217 generates a correction value, and the multiplier 3213 multiplies the generated correction value by the average purchase amount per person and the family structure of the subject, and outputs the result.

The factor determination unit 3216 outputs a correction value 1 to the multipliers 3213 and 3214 when determining that the correction is unnecessary or the factor is unknown.

The adder 3215 adds the output of multiplier 3213 and the output of multiplier 3214 and outputs the result as the corrected nutrient information. As described above, it is possible to correct the purchase amount reduced due to the purchase from other retail stores.

The data processing system 30 according to the second embodiment includes the purchase information correction unit 321 that corrects the data input to the information conversion unit 322, the reception unit 341 receives the purchase information of the customer of the business operator, and the purchase information correction unit 321 corrects purchase information having a purchase amount smaller than a predetermined threshold value by multiplying the purchase information by a correction value. Therefore, the health of a wide range of users can be predicted, and the health of a wider range of subjects can be predicted accurately.

The purchase information correction unit 321 includes: the comparator 3210 that compares the purchase information of the customer of the business operator with the purchase information of a plurality of customers of the business operator; the factor determination unit 3216 that determines, based on the comparison result generated by the comparator 3210, a factor which is the reason why the purchase amount of the purchase information of the customer of the business operator is small; and the correction value generation unit 3217 that generates a correction value corresponding to the determined factor. The purchase information correction unit 321 corrects the purchase information by multiplying the purchase information by the generated correction value. Therefore, it is possible to correct the purchase amount for each estimated factor, make an appropriate correction according to the characteristics of the subject, and accurately predict the health.

### Third Embodiment

In the first embodiment described above, the nutrient information extracted from the purchase information and the nutrient information in the analysis result database 311 are treated in the same manner. Nutrient information extracted from the same type of information can be treated in this way, but in many cases nutrient information is different. Brief-type self-administered diet history questionnaire (BDHQ) is widely used as nutrient information in the health data 210 of the local government. The BDHQ quantitatively examines an ingestion state of nutrients and food based on a questionnaire to the subject. When nutrient information obtained by different methods, such as BDHQ and purchase information, is treated as the same nutrient information, accuracy is reduced. In a third embodiment, a nutrient information comparison and correction unit 323 is provided in the correction unit 320 to solve this problem. In the third embodiment, differences from the first embodiment described above will be mainly described, and descriptions of the same configurations and functions as those of the first embodiment will be omitted.

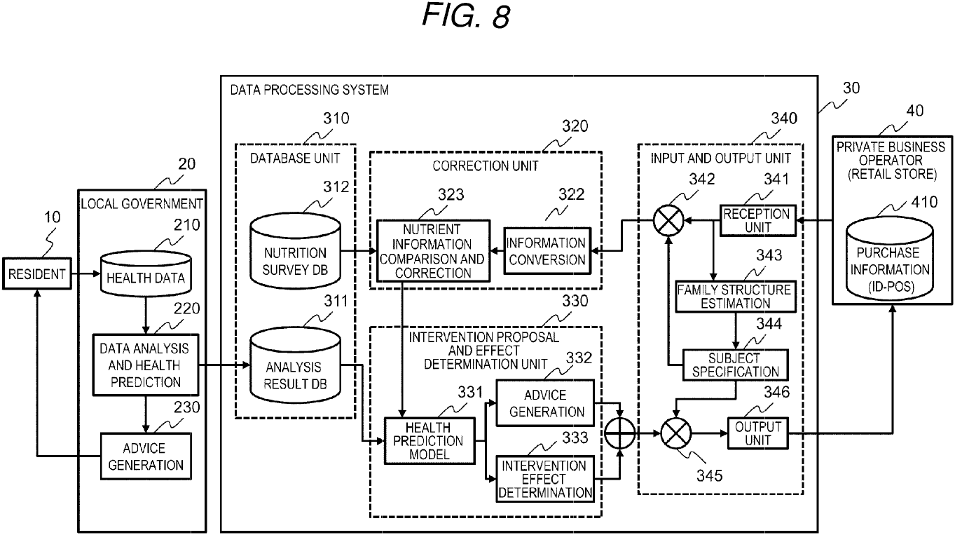

FIG. 8 is a configuration diagram of the data processing system 30 according to the third embodiment.

The data processing system 30 according to the third embodiment includes the database unit 310, the correction unit 320, the intervention proposal and effect determination unit 330, and the input and output unit 340. The database unit 310 includes the analysis result database 311 and a nutrition survey database 312. The nutrition survey database 312 stores nutrition survey data from the Ministry of Health, Labor and Welfare. The nutrition survey database 312 may be configured for online viewing. The correction unit 320 includes the information conversion unit 322 that converts the purchase information into the nutrient information, and the nutrient information comparison and correction unit 323. The intervention proposal and effect determination unit 330 includes the health prediction model 331, the advice generation unit 332, and the intervention effect determination unit 333.

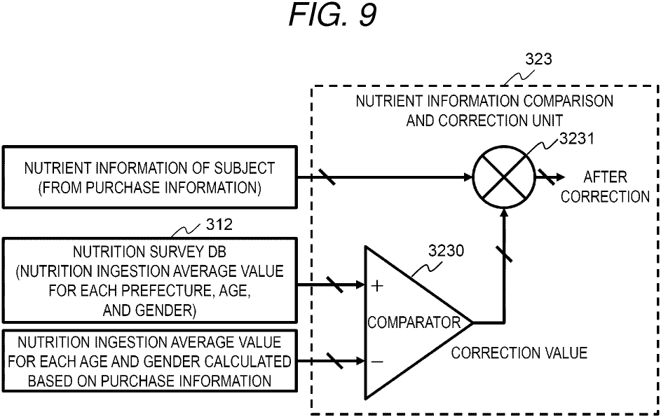

FIG. 9 is a configuration diagram of the nutrient information comparison and correction unit 323 according to the third embodiment.

The nutrient information comparison and correction unit 323 includes a comparator 3230 and a multiplier 3231, and uses a nutrition survey result from the Ministry of Health, Labor and Welfare as a reference value for correction. A nutrition survey is conducted at the BDHQ and is suitable as the reference value. The comparator 3230 compares various ingested nutrients in the nutrition survey result from the Ministry of Health, Labor and Welfare with various ingested nutrients that are calculated based on the purchase information, and calculates, for each of the ingested nutrients, a ratio of the latter to the former (kk1 to kkn, where n is the total number of nutrients). The multiplier 3231 corrects the nutrient information of the subject by multiplying kk1 to kkn as correction values. The nutrition survey result from the Ministry of Health, Labor and Welfare is surveyed for each prefecture, and can be corrected in consideration of regional characteristics.

Although the second embodiment and the third embodiment have been described separately, the second embodiment and the third embodiment may be combined to configure a data processing system including both elements of the second embodiment and the third embodiment.

Since the data processing system 30 according to the third embodiment includes the nutrient information comparison and correction unit 323 that corrects the customer nutrient information input to the health prediction model based on a comparison result between data acquired from the nutrition survey database and the customer nutrient information, the mismatch between the purchase information and the nutrient information can be eliminated, and the prediction accuracy can be improved.

The invention is not limited to the above embodiments and includes various modifications and equivalent configurations within the spirit of the claims. For example, the above embodiments have been described in detail in order to make the invention easy to understand, and the invention is not necessarily limited to those which include all the configurations described. In addition, a part of a configuration of a certain embodiment may be replaced with a configuration of another embodiment. In addition, the configuration of another embodiment may be added to the configuration of the certain embodiment. Further, a part of the configuration of each embodiment may be added to, deleted from, or replaced with another configuration.

Further, parts or all of the configurations, functions, processing units, processing methods described above, and the like may be implemented by the hardware, for example by designing with the integrated circuit, or may be implemented by the software, with a processor to interpret and execute a program that implements each function.

Information such as a program, a table, a file, and the like that implements each function can be stored in a storage device such as a memory, a hard disk, and a solid state drive (SSD), or a recording medium such as an IC card, an SD card, and a DVD.

Control lines and information lines indicate those that are considered necessary for description, and not all the control lines and the information lines necessary for implementation are shown. In fact, it may be considered that almost all the configurations are connected.

What is claimed is:

1. A data processing system comprising:
an arithmetic device configured to execute arithmetic processing;
a storage device accessible by the arithmetic device;
a reception unit configured to receive input data;
an information conversion unit configured to convert the input data;
a health prediction model generated based on nutrient information and an outcome; and
an output unit configured to output information of a subject based on a prediction result generated by the health prediction model, wherein
the reception unit receives the input data relating to a customer of a business operator,
the information conversion unit converts the input data into customer nutrient information indicating a nutrient ingested by the customer,
the health prediction model predicts health based on the customer nutrient information, and
the output unit outputs advice for the health of the subject to the business operator based on the prediction result.

2. The data processing system according to claim 1, further comprising:
a nutrient information comparison and correction unit configured to correct the customer nutrient information input to the health prediction model based on a comparison result between data acquired from a nutrition survey database and the customer nutrient information.

3. The data processing system according to claim 1, further comprising:
an information correction unit configured to correct data input to the information conversion unit, wherein
the reception unit receives purchase information of the customer as the input data, and
the information correction unit corrects purchase information having a purchase amount smaller than a predetermined threshold value by multiplying the purchase information by a correction value.

4. The data processing system according to claim 3, wherein
the information correction unit includes:
a comparator configured to compare the purchase information of the customer with purchase information of a plurality of the customers;
a factor determination unit configured to determine a factor causing a purchase amount of the purchase information of the customer to be small based on a comparison result by the comparator; and
a correction value generation unit configured to generate a correction value corresponding to the determined factor, and
the information correction unit corrects purchase information by multiplying the purchase information by the generated correction value.

5. The data processing system according to claim 1, further comprising:
a nutrient information comparison and correction unit configured to correct the customer nutrient information input to the health prediction model based on a comparison result between data acquired from a nutrition survey database and the customer nutrient information; and
an information correction unit configured to correct data input to the information conversion unit, wherein
the reception unit receives purchase information of the customer as the input data, and
the information correction unit corrects purchase information having a purchase amount smaller than a predetermined threshold value by multiplying the purchase information by a correction value.

6. The data processing system according to claim 1, further comprising:
an analysis result database configured to store an analysis result indicating a relation between nutrient information and an outcome based on an analysis result of health data of a resident, wherein
the health prediction model is generated based on the analysis result stored in the analysis result database.

7. The data processing system according to claim 6, wherein
the health prediction model includes a multiple regression model, and
the analysis result database includes a partial regression coefficient and an intercept of a nutrient of the multiple regression model for the outcome.

8. A data processing method executed by a computer,
the computer including an arithmetic device that executes arithmetic processing and a storage device accessible by the arithmetic device,
the data processing method comprising:
a reception step of receiving input data relating to a customer of a business operator;
an information conversion step of converting the input data into customer nutrient information indicating a nutrient ingested by the customer;
a prediction step of predicting health based on the customer nutrient information using a health prediction model generated based on nutrient information and an outcome; and
an output step of outputting advice for health of the subject to the business operator based on a prediction result obtained in the prediction step.
